(19) United States
(12) Patent Application Publication
(10) Pub. No.: US 2021/0369463 A1
(43) Pub. Date: Dec. 2, 2021

(54) SYSTEMS AND METHODS FOR MANUFACTURING BIOSCAFFOLD EXTRACELLULAR STRUCTURES FOR TISSUE REGENERATION

(71) Applicant: Mentor Worldwide LLC, Irvine, CA (US)

(72) Inventors: Hector Javier Toro Estrella, Irvine, CA (US); Orquidea Helen Garcia, Irvine, CA (US)

(21) Appl. No.: 17/053,419

(22) PCT Filed: May 6, 2019
(86) PCT No.: PCT/US2019/030960

§ 371 Date: Nov. 6, 2020

Related U.S. Application Data
(60) Provisional application No. 62/668,190, filed on May 7, 2018.

(51) Int. Cl. A61F 2/30; A61F 2/28; G16H 10/60; G16H 20/40; G16H 50/70

(52) U.S. Cl. CPC A61F 2/30942; A61F 2/30756; A61F 2/2846; G16H 10/60; A61F 2310/00371; G16H 50/70; A61F 2002/30948; A61F 2002/30985; A61F 2002/30766; G16H 20/40

(57) ABSTRACT

A method of manufacturing a bioscaffold implant for a specific patient is provided. The method can comprise obtaining an image of a tissue section of the specific patient from imaging scans of the tissue section, wherein the tissue section includes a resected portion. The method can further comprise determining on the image of the tissue section a surface topography of the resected portion, determining an image of a bioscaffold implant that matches the surface topography of the resected portion, and manufacturing a bioscaffold implant with a surface portion that mirrors the surface topography of the resected portion.

### CROSS-REFERENCE TO RELATED APPLICATION

This application is an international application claiming the benefit of priority from U.S. Provisional Application No. 62/668,190 filed on May 7, 2018, the entirety of which is incorporated herein by reference.

### FIELD

The embodiments disclosed herein are generally directed towards systems and methods for manufacturing bioscaffold structures. More specifically, there is a need for systems and methods that provide for controlled manufacturing of bioscaffold structures that are designed to promote optimal cellular infiltration and regeneration.

### BACKGROUND

The embodiments disclosed herein are generally directed towards systems and methods that provide for controlled manufacturing of bioscaffold structures that are designed to promote optimal cellular infiltration and regeneration. Bioscaffold structures are usually extracellular constructs that generally are used to replace an organ or tissue on a temporary or permanent basis. The goal of bioscaffold structures can be to aid the restoration of normal function or appearance of the involved organ or tissue. The bioscaffold structure can accomplish this goal by providing a platform from where cells can infiltrate and overtake. This can result in the cells replacing the bioscaffold structure, with their own extracellular matrix structures, while restoring normal organ or tissue function and/or appearance.

To provide a bioscaffold structure suited for optimal cellular infiltration, the bioscaffold generally can include a series of pores that allow for cellular ingrowth. The bioscaffold can also be composed of materials that allow for the requisite biodegradation and bioresorbtion as the cellular ingrowth replaces the bioscaffold and restores organ or tissue function and/or appearance. Those materials, however, should not induce adverse biological responses. The bioscaffold will also have an optimal tensile strength to withstand the stresses of initial implantation and subsequent infiltration. The bioscaffold composition can also possess surface chemistry properties to allow for requisite cell adherence.

Conventional bioscaffolds are manufactured from extracellular matrices (ECM) that are manufactured out of tissue extracted from human cadavers or porcine (pig) products and later decellularized to leave the intact extracellular matrix. When sourced from the dermis (skin) these scaffolds are known as acellular dermal matrices (ADM).

However, these conventional extracellular matrix materials have a number of drawbacks. The materials can trigger immunogenic responses when they are implanted into a patient as the materials may have residual cellular or extracellular components from the donor source. Moreover, since the ADM materials are typically taken from adult or elderly donors, the materials contain high ratios of Collagen I to Collagen III, which provides decreased structural support, cushioning, protection, reinforcement and coverage than tissues containing lower Collagen I to Collagen III ratios.

Besides producing adverse biological responses, conventional bioscaffolds often fail to provide optimal compositions of extracellular matrix proteins (e.g., collagen, elastin, laminin, cytokines, polysaccharides, growth factors, etc.) that help promote cell ingrowth into the bioscaffolds and thus boosting overall tissue regeneration which can help in patient recovery and reducing overall scarring.

Besides failing to provide an optimal mixture of extracellular matrix proteins to promote cell ingrowth and tissue regeneration, conventional ADM bioscaffolds can fail to properly balance the requisite pore size and structure to promote optimal cell and tissue growth and vascularization against providing sufficient structural properties (such as tensile strength and elasticity) required to withstand regular bioscaffold use scenarios such as, for example, suturing and expansion. Moreover, it is apparent that the cellular niche (size, shape, substrate, etc.), rather than the cell itself can ultimately direct and control a cell's fate. As such, the architecture of the cellular niche provided for host cell infiltration will ultimately influence the molecular and mechanical signals that will direct cellular behavior.

Besides an improper balance between promoting optimal cell, tissue growth and vascularization, and providing sufficient structural properties required to withstand regular bioscaffolds use scenarios, current manufacturing methods lack the accuracy and flexibility needed to produce structures having specific and unique properties based on predetermined design criteria.

Moreover, since conventional ADM bioscaffolds are typically come as non-customizable two-sheets of material, they can require extensive handling or manipulation (e.g., cutting, shaping, stitching, etc.) by the surgeon prior to implantation into a patient. This may lead to an increase in risk of contamination to the patient.

Accordingly, there is a need to develop three-dimensional porous extracellular structures and/or bioscaffold structures that minimize host immunogenic response and better promotes cell growth while maintaining sufficient mechanical properties. Moreover, there is a need to precisely control the architecture and size of the produced cell niche, to therefore enable the optimal geometric unit within the extracellular construct to promote cellular infiltration, remodeling and tip cell fate with regenerative response rather than fibrotic response. Furthermore, there is a need to flexibly control the architecture of the construct to enable manufacture multiple versions of the construct that are optimized for particular uses.

### SUMMARY

In one aspect, a method of manufacturing a bioscaffold implant for a specific patient is provided. The method can comprise obtaining an image of a tissue section of the specific patient from imaging scans of the tissue section, wherein the tissue section includes a resected portion. The method can further comprise determining on the image of the tissue section a surface topography of the resected portion, determining an image of a bioscaffold implant that matches the surface topography of the resected portion, and manufacturing a bioscaffold implant with a surface portion that mirrors the surface topography of the resected portion.

In another aspect, a non-transitory computer-readable medium in which a program is stored for causing a computer to perform a method for generating an image of a bioscaffold implant for a specific patient is provided. The method can comprise receiving an image of a tissue section of the specific patient from imaging scans of the tissue section, wherein the tissue section includes a resected portion. The method can further comprise determining a surface topography of the resected portion on the image of the tissue section, and generating an image of a bioscaffold implant with a surface portion that matches the surface topography of the resected portion.

In a further aspect, a system for manufacturing a bioscaffold implant for a specific patient is provided. The system can comprise a computing device. The computing device can comprise a clinical inputs engine configured to receive medical data for a specific patient. The computing device can further comprise an implant configuration engine configured to receive an image of a tissue section of the specific patient from imaging scans of the tissue section, wherein the tissue section includes a resected portion, determine a surface topography of the resected portion on the image of the tissue section, and generate bioscaffold implant design criteria based on the received medical data for the specific patient. The computing device can further comprise an image translation engine configured to generate an image of a bioscaffold implant that matches the surface topography of the resected portion and meets the bioscaffold implant design criteria. The system can further comprise a 3D printer configured to receive the image of the bioscaffold implant generated by the implant configuration engine and produce a manufactured bioscaffold implant with a surface portion that mirrors the surface topography of the resected portion.

### BRIEF DESCRIPTION OF THE DRAWINGS

FIG. 1 is a block diagram that illustrates a computer system, in accordance with various embodiments.

FIG. 2 is a diagram illustrating some example bioscaffold structures, in accordance with various embodiments.

FIG. 3 is a flow chart illustrating a method of manufacturing a bioscaffold implant for a specific patient, in accordance with various embodiments.

FIG. 4 is a flow chart illustrating a method for generating an image of a bioscaffold implant for a specific patient, in accordance with various embodiments.

FIG. 5 is a schematic diagram illustrating a system for manufacturing a bioscaffold implant for a specific patient, in accordance with various embodiments.

FIG. 6 is a flow chart illustrating a method of manufacturing a bioscaffold implant for a specific patient, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Other embodiments, features, objects, and advantages of the present teachings will be apparent from the description and accompanying drawings, and from the claims. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, a tray, a baseplate, a separate metal structure, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

As used herein, “extracellular”, as used in reference to “extracellular material”, “extracellular structure”, “extracellular matrix”, “extracellular construct”, and “extracellular component”, denotes the characteristic of existing outside the cell and can refer to a synthetic or natural material. Examples of materials that are extracellular include synthetic and natural polymers; metabolites; ions; various proteins and non-protein substances (e.g. DNA, RNA, lipids, microbial products, etc.) such as Collagens, Proteoglycans, hormones, growth factors, cytokines, chemokines; various enzymes including, for example, digestive enzymes (e.g., Trypsin and Pepsin), extracellular proteinases (e.g., matrix metalloproteinases, ADAMTSs, Cathepsins) and antioxidant enzymes (e.g., extracellular superoxide dismutase); proteolytic products; extracellular matrix proteins (such as elastin, glycosaminoglycans (GAGs), laminin, fibronectin, etc.), selected cell populations, small molecules and small molecule inhibitors, antibiotics, antimicrobials, nanoparticles, mesoporous silica, silk fibroin, enzymatic degradation sites; anti-fibrotic agents such as anti-transforming growth factor beta (anti-TGF-β) and anti-tumor necrosis factor alpha (anti-TNF-α); pro-angiogenic agents such as vascular endothelial growth factor (VEGF) and placental growth factor (PlGF); and factors affecting adipogenesis and proliferation such as insulin-like growth factor 1 (IGF-1) and Dexamethasone.

As used herein, “bioscaffold” denotes a biocompatible and bioresorbable structure used in tissue engineering that is capable of being implanted in the body in order to promote cell adhesion and tissue regeneration, often for injury recovery. A bioscaffold can be used, for example, in the areas of bone, cartilage, skin, organ, tissue area/volume (e.g., breast tissue), and muscle regeneration.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Unless otherwise defined, scientific and technical terms used in connection with the present teachings described herein shall have the meanings that are commonly understood by those of ordinary skill in the art.

### Computer System

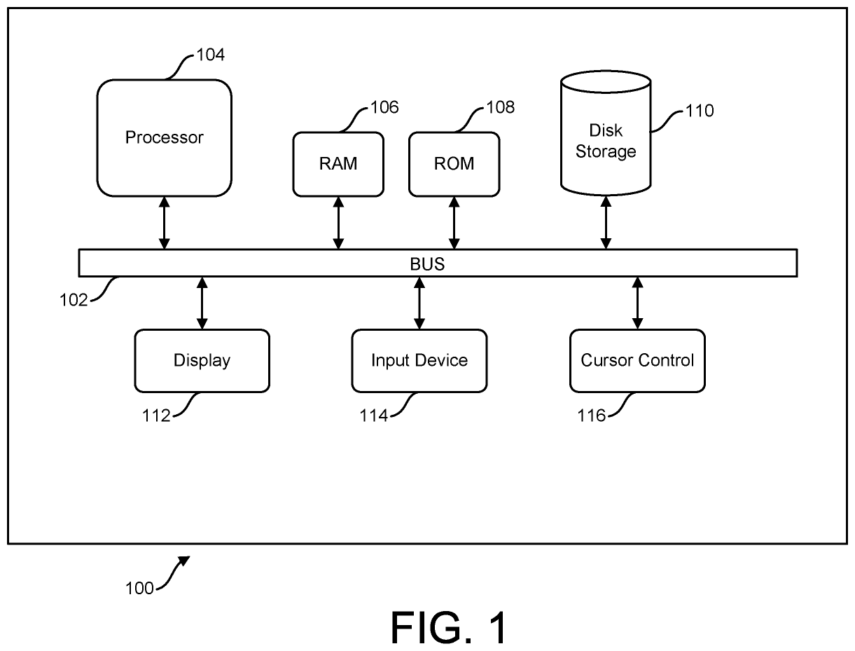

FIG. 1 is a block diagram that illustrates a computer system 100, upon which embodiments, or portions of the embodiments, of the present teachings may be implemented. In various embodiments of the present teachings, computer system 100 can include a bus 102 or other communication mechanism for communicating information, and a processor 104 coupled with bus 102 for processing information. In various embodiments, computer system 100 can also include a memory 106, which can be a random access memory (RAM) or other dynamic storage device, coupled to bus 102 for determining instructions to be executed by processor 104. Memory 106 also can be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communicating information and command selections to processor 104. Another type of user input device is a cursor control 116, such as a mouse, a trackball or cursor direction keys for communicating direction information and command selections to processor 104 and for controlling cursor movement on display 112. This input device 114 typically has two degrees of freedom in two axes, a first axis (i.e., x) and a second axis (i.e., y), that allows the device to specify positions in a plane. However, it should be understood that input devices 114 allowing for 3-dimensional (x, y and z) cursor movement are also contemplated herein.

Consistent with certain implementations of the present teachings, results can be provided by computer system 100 in response to processor 104 executing one or more sequences of one or more instructions contained in memory 106. Such instructions can be read into memory 106 from another computer-readable medium or computer-readable storage medium, such as storage device 110. Execution of the sequences of instructions contained in memory 106 can cause processor 104 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 104 for execution. Such a medium can take many forms, including but not limited to, non-volatile media, volatile media, and transmission media. Examples of non-volatile media can include, but are not limited to, optical, solid state, magnetic disks, such as storage device 110. Examples of volatile media can include, but are not limited to, dynamic memory, such as memory 106. Examples of transmission media can include, but are not limited to, coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 104 of computer system 100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein including flow charts, diagrams and accompanying disclosure can be implemented using computer system 100 as a standalone device or on a distributed network of shared computer processing resources such as a cloud-computing network.

### Structure

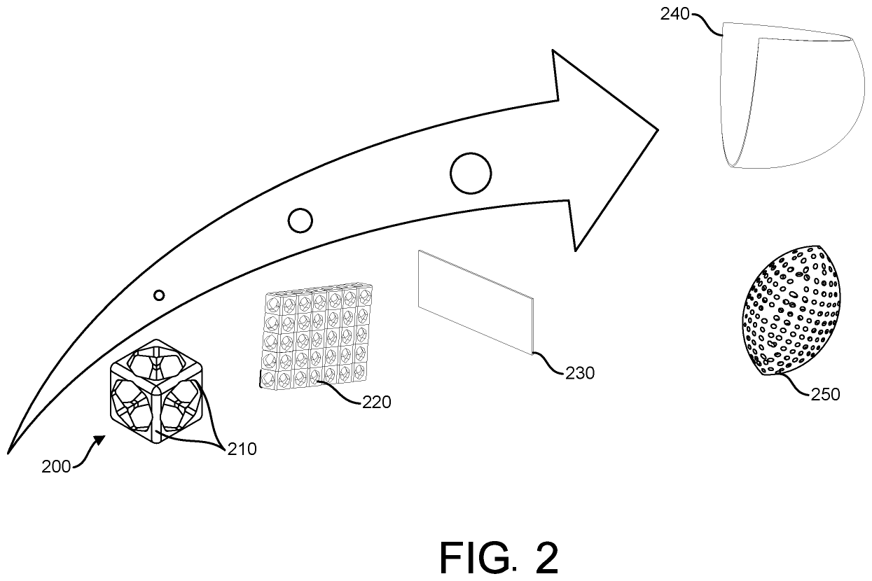

FIG. 2 is a diagram illustrating some example bioscaffold implants, in accordance with various embodiments. Bioscaffold implants can be constructed from bioscaffold structures having a base unit cell structure 200 having a given geometry. Each unit cell can comprise a plurality of filaments 210 composed of, for example, an extracellular material containing, for example, collagen I and collagen III.

A plurality of unit cells 200 (generally composed of a plurality of filaments) can be connected to form a monolayer bioscaffold structure 220. In various embodiments, the plurality of unit cells 200 can also be connected to form a multi-layer structure 200. The plurality of connected unit cells in the bioscaffold structure can also be bioprinted, based on certain needs, to form, for example, a bioscaffold implant shaped as a substantially planar sheet or 3D macrostructure as discussed below.

In accordance with various embodiments, the bioscaffold implant can take many other forms including, for example, membranes, microbeads, fleece, fibers, gels and fiber meshes. A finished product mesh, for example, can provide the requisite porosity to allow optimal cellular infiltration and provide a large enough niche for cells to attach, and ultimately direct cell fate towards a remodeling/regenerative phenotype rather than fibrotic/contractile phenotype. Furthermore, from a mechanical/structural perspective, the arrangement of the unit cell and scaffold structure provides the appropriate mechanical strength and elasticity for the implant to be physiologically relevant as well as useful as a supportive matrix. These features can be provided by the bioscaffold structure using, for example, an optimal extracellular material composition, such as one containing Collagen I and III, thereby providing the necessary structural integrity properties, and just as importantly, the necessary regenerative cues to the infiltrating cells to elicit a regenerative vs. fibrotic response. Without the appropriate mix of ECM proteins, the synthetic scaffold alone may provide the appropriate mechanical properties, but cell surface receptors will not recognize polymer as self and regenerate. Determining the optimal composition to provide the requisite integrity and tissue/cell regenerative properties can be accomplished by systemically varying, for example, pore size, angular filament deposition range, density, height, polymer type and filament size (e.g., diameter).

Returning to FIG. 2, in accordance with various embodiments, the final tissue construct of the bioscaffold implant can be, for example, a flat sheet 230 that is two-dimensional. As also provided by way of example in FIG. 2, another and more advanced final tissue construct of the bioscaffold implant can be, for example, molded sheets/constructs 240 that provide a “hand in glove” fit for tissue reinforcement, breast implants or other medical devices, anatomy or physiology within the breast (e.g., breast pocket), other organs, or other anatomy. As also provided by way of example in FIG. 2, yet another advanced final tissue construct of the bioscaffold implant can be, for example, a “ready-to-use” lumpectomy defect implant 250 within the breast (e.g., breast pocket), other organs, or other anatomy. The solid, implant-style construct can be printed, for example, from a range of from about 20 g to about 4500 g sizes for breast applications (e.g., lumpectomy) or custom sized for other anatomic applications. For example, the implant can be a prolate spheroid shaped (i.e. football) (as exemplary illustrated in FIG. 2), custom shaped, or shaped in another pre-determined geometrical configuration.

Such final tissue constructs of bioscaffold implants advantageously provide improved ease of use during the implant procedure by minimizing intraoperative manipulation while improve procedural efficiency for patients. For example, a three-dimensional (3D) construct, in accordance with various embodiments, would provide patients with a ‘ready-to-implant’ option, improving procedural efficiency for physicians and patients in the areas of, for example, tissue reinforcement and lumpectomy implants. By contrast, current cadaveric acellular dermal matrix (ADM) offerings are supplied as two-dimensional (2D) sheets of tissue, requiring extensive manipulation of the tissue sheet necessitating the surgeon to sew (or bind or connect) the acellular dermal matrix sheet into a pouch-like structure prior to implantation to create an adequate 3D pocket for a breast implant or tissue reinforcement application. This extensive manipulation requires additional procedure time and introduces potential contamination.

Moreover, ADMs are hampered by tissue quality (age, smoking history, drug use, etc.), varied national regulatory constraints, donor availability, donor matching, host immune status, cost, and so on. By contrast, bioprinted scaffolds, in accordance with various embodiments herein, advantageously eliminates issues of donor availability, variability and quality/health status, tissue quality and regulatory policies. Moreover, by being bioprinted using controlled parameters (discussed in detail below) of the scaffold to meet required properties of the end product bioscaffold implant, the bioprinted scaffolds allow for personalized medical applications that ADMs simply do not. These personalized medical applications can include, for example only and not limited to, lumpectomies (as discussed above), tubular cartilage applications, valvular heart disease applications, and coronary artery disease applications, hernia repair applications, tissue graft applications, venous, arterial and lymphatic vessel applications, structural applications or supportive applications where soft tissue defects exist.

With regards to tissue reinforcement, the uses and advantages of a biologic acellular dermal matrix, in accordance with various embodiments, are varied and substantial. One exemplary use is attachment to the inferior border of the pectoralis muscle, thus allowing for greater initial tissue expander fill volume for a two-stage breast reconstruction or implant placement for a single stage breast reconstruction. Another exemplary use is implant position maintenance (support) by helping to define the shape of the breast pocket by defining the inframammary fold, supporting the implant in a pre-pectoral breast reconstruction, and correcting implant malposition such as symmastia, bottoming out, etc. Another exemplary use is aesthetic defect camouflaging by using the scaffold as a buffer or a means to thicken tissue to mask unwanted cosmetic outcomes such as rippling. Other exemplary uses and benefits are capsular contracture reduction and more positive tissue response during radiation treatments.

Additionally, currently available tissue offerings may have inconsistent surface topography throughout. In the case of woven, spun or knitted synthetic constructs, the lack of appropriate microarchitecture and/or an optimized collagen and ECM material composition for the construct affects the ability of host cells to recognize the graft as self, and promote cell adhesion, and thus inhibit a robust regenerative response. By controlling the combination of components in the scaffold to provide specific physical properties and implant outcomes, in accordance with the various embodiments herein, the construct will have consistent surface topography throughout with an engineered microarchitecture (controlling microarchitecture properties such as, for example, porosity, fiber diameter, spacing, height of matrix, fiber orientation, etc.) that provides the appropriate scaffold for a robust wound healing, regenerative, infiltrative and remodeling response.

Moreover, the construct will provide, for example, cushioning and structural support for other tissues, supplemental support, protection, reinforcement and covering within the breast, other organs, or other anatomy and surrounding tissue, while stimulating host cell remodeling. The construct will allow, for example, plastic and reconstructive surgeons to support, repair, elevate and reinforce deficiencies where weakness or voids exist in, for example, the breast, other organs, or other anatomy and surrounding tissue that requires the addition of material to obtain the desired surgical outcome. Furthermore, the construct will allow for the repair of fascial defects within the breast, other organs, or other anatomy and surrounding tissues that require the addition of a reinforcing or bridging material to obtain a desired surgical result.

Additive manufacturing processes, such as, for example, 3D printing manufacturing methods, allow for control of the macro (overall finished shape) and micro (cell units) structure of the construct, and will be discussed in detail below. The manufacturing process of 3D bioprinting, for example, allows for the flexible and accurate production of all final product tissue construct configurations, without the limitation of traditional manufacturing limitations such as tool access, allowing for ultimate design freedom of the complex unit cell geometries needed. These processes, combined with sophisticated software methods that can incorporate specific design criteria and specific medical data of a patient, provide systems that allow for even greater flexibility and accurate production of bioscaffold implants personalized for specific patients. These systems and methods are discussed in detail below.

### Methods

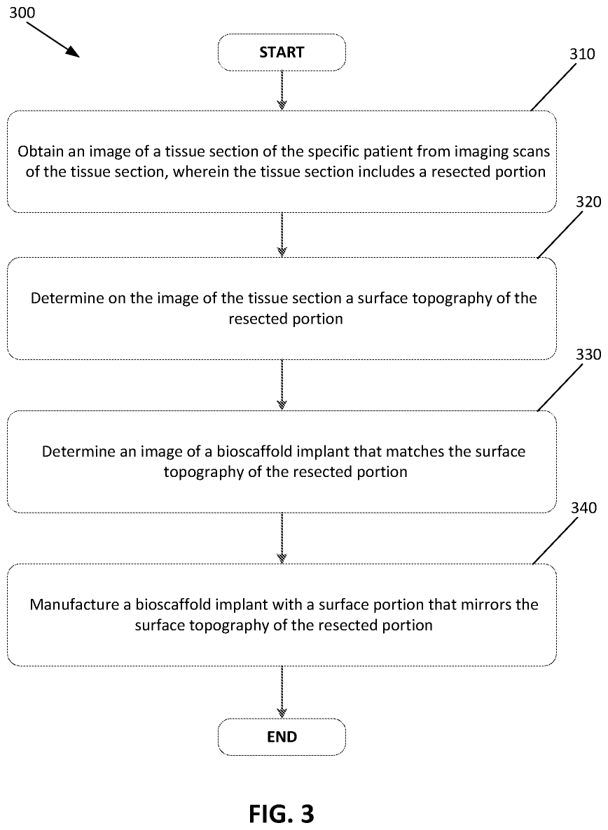

FIG. 3 is a flow chart illustrating a method 300 of manufacturing a bioscaffold implant for a specific patient, in accordance with various embodiments. Method 300 is illustrative only and embodiments can use variations of method 300.

In step 310 of method 300 of FIG. 3, an image of a tissue section of the specific patient is obtained from imaging scans of the tissue section, wherein the tissue section includes a resected portion (i.e., void volume or scar left on the tissue section after the resected tissue is removed). Note that the resected tissue removed from the resected portion can be or also be, for example but not limited to, an anatomical defect or abnormality to be corrected. In accordance with various embodiments, the image can be two-dimensional. The image can also be three-dimensional. The imaging scans can originate from any source known in the art. Examples include, but are not limited to, magnetic resonance imaging (MRI), computed tomography scan (CT scan), basic x-ray, ultrasound, nuclear medicine imaging (including positron-emission tomography [PET]), nuclear medicine, elastography, tomography, echocardiography, functional near-infrared spectroscopy, magnetic particle imaging, photographs, etc. In various embodiments, the image obtained can be of the resected tissue that is removed from the resected portion.

In step 320 of method 300 of FIG. 3, a surface topography of the resected portion on the image of the tissue section is determined. Again, note that, in accordance with various embodiments herein, the resected tissue removed from the resected portion can be or also be, for example but not limited to, an anatomical defect or abnormality to be corrected. In various embodiments, the surface topography that is determined can be of the resected tissue that is removed from the resected portion.

In step 330 of method 300 of FIG. 3, an image of a bioscaffold implant is determined that matches the surface topography of the resected portion. In various embodiments, the image of the bioscaffold implant that is determined matches the surface topography of the resected tissue that is removed from the resected portion.

In step 340 of method 300 of FIG. 3, a bioscaffold implant is manufactured with a surface portion that mirrors the surface topography of the resected portion. In various embodiments, the bioscaffold implant can be manufactured with a surface portion that matches the surface topography of the resected tissue that is removed from the resected portion.

In accordance with various embodiments, the method of manufacturing a bioscaffold implant for a specific patient, such as method 300 of FIG. 3, can further include receiving design criteria for the bioscaffold implant, and determining an image of a bioscaffold implant that satisfies the design criteria. Such steps can assist in, for example, step 330 of method 300 of FIG. 3 in accurately determining an image of a bioscaffold implant that matches the surface topography of the resected portion (or resected tissue). Further, such steps can assist in generating an implant image designed to not only match the surface topography of the resected portion, but also possess properties unique to the specific patient and properties that enable a biologically, structurally and mechanically robust implant design for subsequent manufacturing.

In accordance with various embodiments, the method of manufacturing a bioscaffold implant for a specific patient, such as method 300 of FIG. 3, can further include receiving medical data for the specific patient, generating design criteria for the bioscaffold implant based on the received medical data for the specific patient, and determining an image of a bioscaffold implant that satisfies the design criteria. Similar to above, such steps can assist in generating a bioscaffold implant image designed to not only match the surface topography of the resected portion (or resected tissue), but also possess properties unique to the specific patient and properties that enable a biologically, structurally and mechanically robust implant design for subsequent manufacturing. Furthermore, such steps allow, for example, an associated software program to generate design criteria even more personalized to a specific patient by using the received medical data of the specific patient to generate design criteria specific to the needs of the patient and taking into account specific attributes of the patient. Examples of such medical data types, design criteria types, and associated software programs will the discussed in detail below.

Regarding medical data for a specific patient, in accordance with various embodiments, the medical data can include, for example, patient demographics, procedure type, surgical information, medical history, patient physical data, and combinations thereof.

Patient demographic data can include many various types of patient specific demographic data. This data can include, for example, patient age, height, bodyweight, BMI, and race.

Procedure type data can include many various types of patient specific procedure related data, with the data collected capable of being adjusted depending on the base procedure desired. For example, types of procedures can include primary breast augmentation, revision augmentation, primary reconstruction, revision reconstruction or lumpectomy. Surgery type data can include, for example, breast reduction, pocket adjustment, skin adjustment, scar revision, etc. If a reconstruction is desired, further data can include, for example, choice between delayed or immediate procedure, choice between direct to implant (single stage) or a two-stage procedure, consideration of any other therapies in and around the time of the procedure (radiation, chemotherapy, hormone, or other relevant procedure), and considerations if a mastectomy is desired (weight, type and flap use). Location of procedure (left or right breast, for example) can be relevant. Necessity of additional procedures (e.g., mastopexy, etc.) can be also relevant. The procedure type data can also include identifying the necessity for specific devices for certain particular medical events such as, for example, asymmetry, breast tissue atrophy, chest wall deformity, etc.

Surgical information data can include many types of data including, for example, incision size, incision location (e.g., inframammary, transaxially, periareolar, mastectomy scar, etc.), and device placement (e.g., submuscular, subglandular, etc.).

Medical history data can include many types of data including, for example, noted conditions that can compromise or complicate wound healing, smoking status, diabetes status, immune status, hypertension, high cholesterol, OBGYN history (breast cancer, familial breast cancer, related pathological details, etc.), noted demonstrated patient tissue characteristics that are incompatible, noted potential unwanted surgical risk related to treatment for a condition, previous device history (e.g., noted previous implant, implant type—e.g., acellular dermal matrix or synthetic, previous expander), etc.

Patient physical data can include many types of data including, for example, which breast to be operated on, any noted anatomic or physiological abnormalities, current breast size, expected breast size, additional pre-op measurements (e.g., not part of the CT scan), etc.

Regarding design criteria, in accordance with various embodiments, the design criteria can include, for example, biological parameters, structural parameters, mechanical parameters, and combinations thereof.

In accordance with various embodiments, the design criteria can comprise biological parameters. The biological parameters can include, for example but not limited to, cellular infiltration or attachment type, collagen synthesis type, vascularization type, incorporation profile, and combinations thereof. In accordance with various embodiments, the biological parameters can comprise attachment type. The biological parameters can comprise collagen synthesis type. The biological parameters can comprise vascularization type. The biological parameters can comprise incorporation profile.

In accordance with various embodiments, the design criteria can comprise structural parameters. The structural parameters can include, for example but not limited to, protein composition, protein concentration, surface topography, pore size, filament size, construct thickness, fenestration count, hydration level, macro size and combinations thereof.

In accordance with various embodiments, the structural parameters can comprise protein composition. The protein composition can comprise Collagen I and Collagen III. The protein concentration can include a Collagen I to Collagen III ratio similar to those contained within human dermis (e.g., fetal, adolescent, adult and elderly) as shown in Table I.

TABLE I

| Dermis Type | Collagen I/III Ratio |
|---|---|
| Fetus | 0.95 ± 0.03 |
| Adolescent | 2.27 ± 0.13 |
| Adult | 2.46 ± 0.15 |
| Elderly | 2.97 ± 0.40 |

That is, in various embodiments, the extracellular material can have a Collagen I to Collagen III ratio in the range of between about 0.5 to about 3.5, or in the range of between about 0.75 to about 3.0, or in the preferred range of between about 0.9 to about 2.5. The preferred Collagen I to Collagen III ratio range of between about 0.9 to about 2.5 may offer advantages against conventional tissue offerings, which are predominantly taken from adult and elderly donors, contain a high ratio (i.e., greater than about 2.4) of Collagen I to Collagen III, thereby providing decreased support, cushioning, protection, reinforcement and covering than do tissues containing lower ratios.

More specific ratios of Collagen I to Collagen III can include about 0.5, about 0.55, about 0.6, about 0.65, about 0.7, about 0.75, about 0.8, about 0.85, about 0.9, about 0.95, about 1.0, about 1.05, about 1.1, about 1.15, about 1.2, about 1.25, about 1.3, about 1.35, about 1.4, about 1.45, about 1.5, about 1.55, about 1.6, about 1.65, about 1.7, about 1.75, about 1.8, about 1.85, about 1.9, about 1.95, about 2.0, about 2.05, about 2.1, about 2.15, about 2.2, about 2.25, about 2.3, about 2.35, about 2.4, about 2.45, about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, about 3.3, about 2.35, about 3.4, about 3.45, about 3.5 and ranges between any two of these values.

In accordance with various embodiments, the structural parameters can comprise surface topography. The structural parameters can comprise pore size. The pore size can be between about 100 microns to about 500 microns. The structural parameters can comprise filament size. The filament diameter can be less than about 100 microns.

Further, in accordance with various embodiments, the structural parameters can comprise construct thickness. The structural parameters can comprise fenestration count (e.g., fenestration number, orientation and location/arrangement). The structural parameters can comprise hydration level. The structural parameters can comprise macro size/configuration (e.g., size of the implant).

In accordance with various embodiments, the design criteria can comprise mechanical parameters. The mechanical parameters can include, for example but not limited to, tensile strength, stiffness level, max load level, tensile stress, tensile strain, modulus of elasticity, and combinations thereof.

In accordance with various embodiments, the mechanical parameters can comprise tensile strength. The tensile strength can between about 10 to about 200 Newtons per centimeter (N/cm). Further, the tensile strength is between about 30 to about 100 Newtons per centimeter. Preferably, the tensile strength is between about 50 to about 85 Newtons per centimeter.

In accordance with various embodiments, the mechanical parameters can comprise stiffness level. The stiffness level can be less than about 80 Newtons per millimeter. Further, the stiffness level can be less than about 18 Newtons per millimeter. Preferably, the stiffness level can be greater than about 5 Newtons per millimeter and less than about 18 Newtons per millimeter.

In accordance with various embodiments, the mechanical parameters can comprise max load level. The max load level can be greater than about 50 Newtons. Further, the max load level can be greater than about 150 Newtons. Preferably, the max load level can be greater than about 150 Newtons and less than 400 Newtons.

In accordance with various embodiments, the mechanical parameters comprise tensile stress. The tensile stress can be between about 3 to about 100 megapascals. Preferably, the tensile stress can be between about 10 to about 30 megapascals.

In accordance with various embodiments, the mechanical parameters comprise tensile strain (a ratio of the extension and original length of the bioscaffold structure). The tensile strain can be greater than about 10 percent. Further, the tensile strain can be greater than about 35 percent. Preferably, the tensile strain can be from about 20 to about 80 percent.

In accordance with various embodiments, the mechanical parameters comprise modulus of elasticity. The modulus of elasticity can be from about 10 to about 450 megapascals. Further, the modulus of elasticity can be less than about 150 megapascals. Preferably, the modulus of elasticity can be from about 60 to about 150 megapascals.

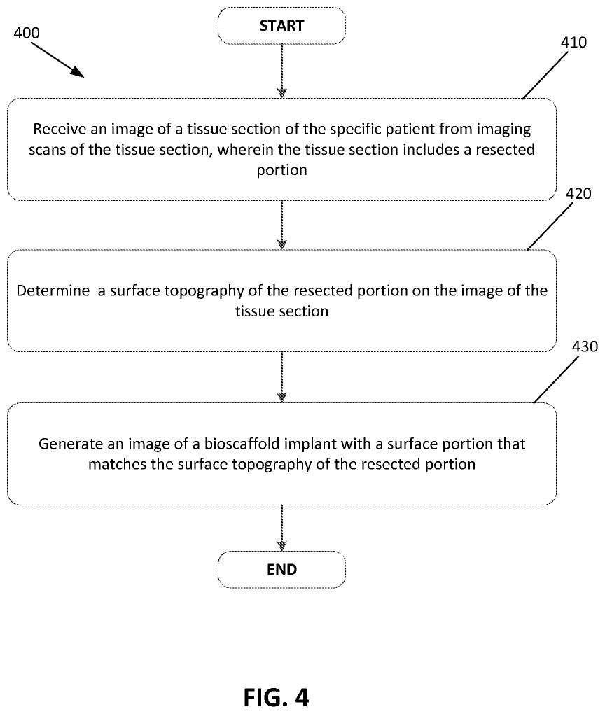

As described above, methods described herein such as, for example, method 300, can be implemented using computer system 100 as a standalone device or on a distributed network of shared computer processing resources such as a cloud-computing network. As such, a non-transitory computer-readable medium can be provided in which a program is stored for causing a computer to perform the disclosed methods for generating an image of a bioscaffold implant for a specific patient. See below in reference to FIG. 4 for additional discussion.

It should also be understood that the preceding embodiments can be provided, whole or in part, as a system of components integrated to perform the methods described. For example, the workflow of FIG. 3 can be provided as, or on, a system of components or stations for generating an image of a bioscaffold implant for a specific patient.

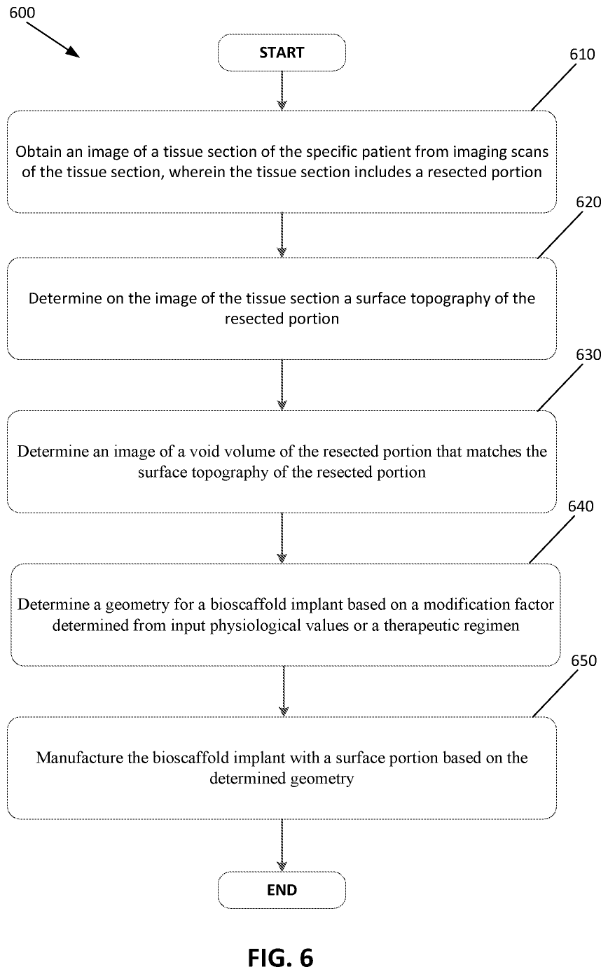

FIG. 6 is a flow chart illustrating a method 600 of manufacturing a bioscaffold implant for a specific patient, in accordance with various embodiments. Method 600 is illustrative only and embodiments can use variations of method 600. For example, method 600 may be an alternative embodiment of method 300, as described above.

In step 610 of method 600 of FIG. 6, an image of a tissue section of the specific patient is obtained from imaging scans of the tissue section, wherein the tissue section includes a resected portion. Note that the resected tissue removed from the resected portion can be or also be, for example but not limited to, an anatomical defect or abnormality to be corrected. In accordance with various embodiments, the image can be two-dimensional. The image can also be three-dimensional. The imaging scans can originate from any source known in the art. Examples include, but are not limited to, magnetic resonance imaging (MRI), computed tomography scan (CT scan), basic x-ray, ultrasound, nuclear medicine imaging (including positron-emission tomography [PET]), nuclear medicine, elastography, tomography, echocardiography, functional near-infrared spectroscopy, magnetic particle imaging, photographs, etc.

In step 620 of method 600 of FIG. 6, a surface topography (and general architecture) of the resected portion on the image of the tissue section is determined. Again, note that, in accordance with various embodiments herein, the resected tissue removed from the resected portion can be or also be, for example but not limited to, an anatomical defect or abnormality to be corrected.

In step 630 of method 600 of FIG. 6, an image of a void volume of the resected portion is determined that matches the surface topography (and general architecture) of the resected portion.

In step 640 of method 600 of FIG. 6, a geometry for a bioscaffold implant is determined based on a modification factor determined from input physiological values or a therapeutic regimen. For example, the modification factor may include a mathematical model. Key inputs into the proposed algorithm include, but are not limited to, size of the excision, breast size, radiation including radiation boost, increasing number of radiation fields, and total radiation dose, radiation technique and use of chemotherapy. Furthermore, an over or under correction of about 1%, about 2%, about 3%, about 4%, about 5%, about 6%, about 7%, about 8%, about, 9%, about 10%, about 11%, about 12%, about 13%, about 14%, about 15%, about 16%, about 17%, about 18%, about 19% or about 20% may be required to account for late contracture that often occurs in irradiated wounds. The input physiological values may be a medical history of the specific patient. For example, the medical history may include an age of the specific patient or an age of the void volume. The therapeutic regimen may include past and future chemotherapy or radiation therapy. It is understood that other therapies and/or treatments that change a morphological nature of tissue may also be included.

In step 650 of method 600 of FIG. 6, a bioscaffold implant is manufactured with a surface portion based on the determined geometry.

According to aspects, method 600 may further include running a simulation based on historical clinical data to determine the geometry. According to aspects, method 600 may further include implanting the bioscaffold implant in the specific patient in a patient procedure.

Referring now to FIG. 4, FIG. 4 is a flow chart illustrating an example non-transitory computer-readable medium in which a program is stored for causing a computer to perform a method 400 for generating an image of a bioscaffold implant for a specific patient, in accordance with various embodiments. Method 400 is illustrative only and embodiments can use variations of method 400.

In step 410 of method 400 of FIG. 4, an image of a tissue section of the specific patient is received from imaging scans of the tissue section, wherein the tissue section includes a resected portion. In accordance with various embodiments, the image can be two-dimensional. The image can also be three-dimensional. The imaging scans can originate from any source known in the art. Examples include, but are not limited to, magnetic resonance imaging (MRI), computed tomography scan (CT scan), basic x-ray, ultrasound, nuclear medicine imaging (including positron-emission tomography [PET]), nuclear medicine, elastography, tomography, echocardiography, functional near-infrared spectroscopy, magnetic particle imaging, photographs, etc.

In step 420 of method 400 of FIG. 4, a surface topography of the resected portion on the image of the tissue section is determined.

In step 430 of method 400 of FIG. 4, an image of a bioscaffold implant is determined with a surface portion that matches the surface topography of the resected portion.

In accordance with various embodiments, the non-transitory computer-readable medium in which a program is stored for causing a computer to perform a method for generating an image of a bioscaffold implant for a specific patient, such as method 400 of FIG. 4, can further include receiving design criteria for the bioscaffold implant, and determining an image of a bioscaffold implant that satisfies the design criteria. Such steps can assist in, for example, step 430 of method 400 of FIG. 4 in accurately determining an image of a bioscaffold implant with a surface portion that matches the surface topography of the resected portion. Further, such steps can assist generating an implant image designed to not only match the surface topography of the resected portion, but also possess properties unique to the specific patient and properties that enable a biologically, structurally and mechanically robust implant design for subsequent manufacturing.

In accordance with various embodiments, the non-transitory computer-readable medium in which a program is stored for causing a computer to perform a method for generating an image of a bioscaffold implant for a specific patient, such as method 400 of FIG. 4, can further include receiving medical data for the specific patient, generating design criteria for the bioscaffold implant based on the received medical data for the specific patient, and determining an image of a bioscaffold implant that satisfies the design criteria. Similar to above, such steps can assist in generating a bioscaffold implant image designed to not only match the surface topography of the resected portion, but also possess properties unique to the specific patient and properties that enable a biologically, structurally and mechanically robust implant design for subsequent manufacturing. Furthermore, such steps allow, for example, the computer to generate design criteria even more personalized to a specific patient by using the received medical data of the specific patient to generate design criteria specific to the needs of the patient and taking into account specific attributes of the patient. Examples of such medical data types, design criteria types, and associated software programs will the discussed in detail below.

Regarding medical data for a specific patient, in accordance with various embodiments, the medical data can include, for example, patient demographics, procedure type, surgical information, medical history, patient physical data, and combinations thereof.

Patient demographic data can include many various types of patient specific demographic data. This data can include, for example, patient age, height, and bodyweight, BMI, race, etc.

Procedure type data can include many various types of patient specific procedure related data, with the data collected capable of being adjusted depending on the base procedure desired. For example, types of procedures can include primary breast augmentation, revision augmentation, primary reconstruction, revision reconstruction or lumpectomy. Surgery type data can include, for example, breast reduction, pocket adjustment, skin adjustment, scar revision, etc. If a reconstruction is desired, further data can include, for example, choice between delayed or immediate procedure, choice between direct to implant (single stage) or a two-stage procedure, consideration of any other therapies in and around the time of the procedure (radiation, chemotherapy, hormone, or other relevant procedure), and considerations if a mastectomy is desired (weight, type and flap use). Location of procedure (left or right breast, for example) can be relevant. Necessity of additional procedures (e.g., mastopexy, etc.) can be also relevant. The procedure type data can also include identifying the necessity for specific devices for certain particular medical events such as, for example, asymmetry, breast tissue atrophy, chest wall deformity, etc.

Surgical information data can include many types of data including, for example, incision size, incision location (e.g., inframammary, transaxially, periareolar, mastectomy scar, etc.), and device placement (e.g., submuscular, subglandular, etc.).

Medical history data can include many types of data including, for example, noted conditions that can compromise or complicate wound healing, smoking status, diabetes status, immune status, hypertension, high cholesterol, OBGYN history (breast cancer, familial breast cancer, related pathological details, etc.), noted demonstrated patient tissue characteristics that are incompatible, noted potential unwanted surgical risk related to treatment for a condition, previous device history (e.g., noted previous implant, implant type—e.g., acellular dermal matrix or synthetic, previous expander), etc.

Patient physical data can include many types of data including, for example, which breast to be operated on, any noted anatomic or physiological abnormalities, current breast size, expected breast size, additional pre-op measurements (e.g., not part of the CT Scan), etc.

Regarding design criteria, in accordance with various embodiments, the design criteria can include, for example, biological parameters, structural parameters, mechanical parameters, and combinations thereof.

In accordance with various embodiments, the design criteria can comprise biological parameters. The biological parameters can include, for example but not limited to, cellular infiltration or attachment type, collagen synthesis type, vascularization type, incorporation profile, and combinations thereof. In accordance with various embodiments, the biological parameters can comprise attachment type. The biological parameters can comprise collagen synthesis type. The biological parameters can comprise vascularization type. The biological parameters can comprise incorporation profile.

In accordance with various embodiments, the design criteria can comprise structural parameters. The structural parameters can include, for example but not limited to, protein composition, protein concentration, surface topography, pore size, filament size, construct thickness, fenestration count, hydration level, macro size and combinations thereof.

In accordance with various embodiments, the structural parameters can comprise protein composition. The protein composition can comprise Collagen I and Collagen III. The protein concentration can include a Collagen I to Collagen III ratio similar to those contained within human dermis (e.g., fetal, adolescent, adult and elderly) as shown in Table I (see above).

That is, in various embodiments, the protein composition can have a Collagen I to Collagen III ratio in the range of between about 0.5 to about 3.5, or in the range of between about 0.75 to about 3.0, or in the preferred range of between about 0.9 to about 2.5. The preferred Collagen I to Collagen III ratio range of between about 0.9 to about 2.5 may offer advantages against conventional tissue offerings, which are predominantly taken from adult and elderly donors, contain a high ratio (i.e., greater than about 2.4) of Collagen I to Collagen III, thereby providing decreased support, cushioning, protection, reinforcement and covering than do tissues containing lower ratios.

More specific ratios of Collagen I to Collagen III can include about 0.5, about 0.55, about 0.6, about 0.65, about 0.7, about 0.75, about 0.8, about 0.85, about 0.9, about 0.95, about 1.0, about 1.05, about 1.1, about 1.15, about 1.2, about 1.25, about 1.3, about 1.35, about 1.4, about 1.45, about 1.5, about 1.55, about 1.6, about 1.65, about 1.7, about 1.75, about 1.8, about 1.85, about 1.9, about 1.95, about 2.0, about 2.05, about 2.1, about 2.15, about 2.2, about 2.25, about 2.3, about 2.35, about 2.4, about 2.45, about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, about 3.3, about 2.35, about 3.4, about 3.45, about 3.5 and ranges between any two of these values.

In accordance with various embodiments, the structural parameters can comprise surface topography. The structural parameters can comprise pore size. The pore size can be between about 100 microns to about 500 microns. The structural parameters can comprise filament size. The filament diameter can be less than about 100 microns.

Further, in accordance with various embodiments, the structural parameters can comprise construct thickness. The structural parameters can comprise fenestration count (e.g., fenestration number, orientation and location/arrangement). The structural parameters can comprise hydration level. The structural parameters can comprise macro size (e.g., size of implant)

In accordance with various embodiments, the design criteria can comprise mechanical parameters. The mechanical parameters can include, for example but not limited to, tensile strength, stiffness level, max load level, tensile stress, tensile strain, modulus of elasticity, and combinations thereof.

In accordance with various embodiments, the mechanical parameters can comprise tensile strength. The tensile strength can between about 10 to about 200 Newtons per centimeter (N/cm). Further, the tensile strength is between about 30 to about 100 Newtons per centimeter. Preferably, the tensile strength is between about 50 to about 85 Newtons per centimeter.

In accordance with various embodiments, the mechanical parameters can comprise stiffness level. The stiffness level can be less than about 80 Newtons per millimeter. Further, the stiffness level can be less than about 18 Newtons per millimeter. Preferably, the stiffness level can be greater than about 5 Newtons per millimeter and less than about 18 Newtons per millimeter.

In accordance with various embodiments, the mechanical parameters can comprise max load level. The max load level can be greater than about 50 Newtons. Further, the max load level can be greater than about 150 Newtons. Preferably, the max load level can be greater than about 150 Newtons and less than 400 Newtons.

In accordance with various embodiments, the mechanical parameters comprise tensile stress. The tensile stress can be between about 3 to about 100 megapascals. Preferably, the tensile stress can be between about 10 to about 30 megapascals.

In accordance with various embodiments, the mechanical parameters comprise tensile strain. The tensile strain can be greater than about 10 percent. Further, the tensile strain can be greater than about 35 percent. Preferably, the tensile strain can be from about 20 to about 80 percent.

In accordance with various embodiments, the mechanical parameters comprise modulus of elasticity. The modulus of elasticity can be from about 10 to about 450 megapascals. Further, the modulus of elasticity can be less than about 150 megapascals. Preferably, the modulus of elasticity can be from about 60 to about 150 megapascals.

As described herein, computer implemented methods described herein such as, for example, method 400, can be implemented using computer system 100 as a standalone device or on a distributed network of shared computer processing resources such as a cloud-computing network.

It is understood that the preceding embodiments can be provided, whole or in part, as a system of components integrated to perform the methods described. For example, the workflow of FIG. 4 can be provided as, or on, a system of components or stations for generating an image of a bioscaffold implant for a specific patient.

### Systems

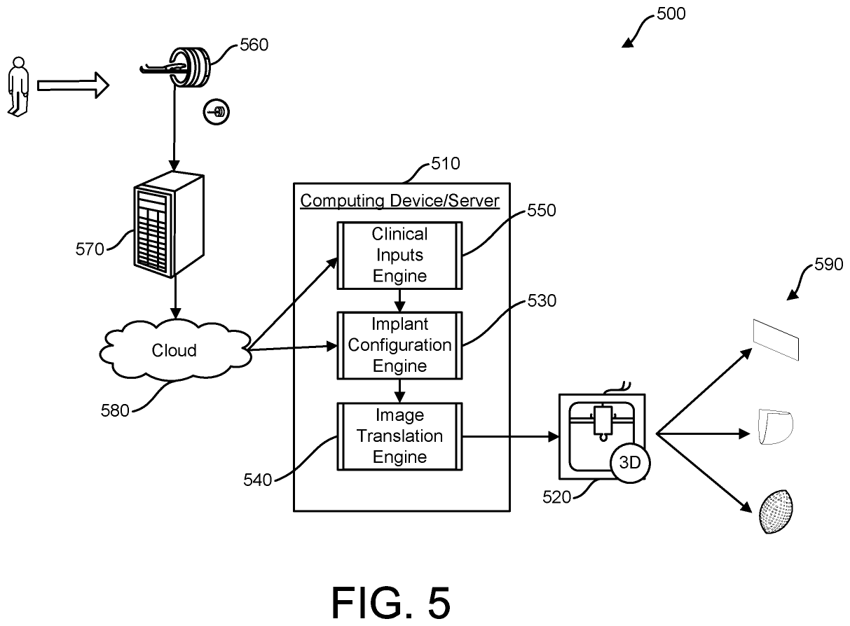

In accordance with various embodiments, FIG. 5 is a schematic diagram illustrating one example of a system for manufacturing a bioscaffold implant for a specific patient. As depicted herein, a system 500 is provided that can include a computing device or server 510 and a bio-printer (or 3D printer) 520.

Computing device or server 510 (hereinafter computing device 510) can include an implant configuration engine 530 configured and arranged to, for example, receive an image of a tissue section of the specific patient from imaging scans of the tissue section, wherein the tissue section includes a resected portion, and determine a surface topography (and general architecture) of the resected portion on the image of the tissue section, and receive design criteria for the specific patient. Computing device 510 can further include an image translation engine 540 configured and arranged to generate an image of a bioscaffold implant that matches the surface topography (and general architecture) of the resected portion and meets the bioscaffold implant design criteria.

3D printer 520 can be configured to receive the image of the bioscaffold implant generated by the implant translation engine 540 and produce a manufactured bioscaffold implant 590 with a surface portion that mirrors the surface topography of the resected portion. 3D printer 520 can receive such bioscaffold implant image data from image translation engine 540, which can also be configured to translate its image data output into a language readable by 3D printer 520.

The manufactured bioscaffold implant can take many forms as is necessary to meet the design criteria set forth, to thus meet the specific needs of the specific patient requiring the bioscaffold implant. As discussed above, with reference to FIG. 2, and in accordance with various embodiments, manufactured bioscaffold implant 590 can be, for example only and in no way limited to these constructs, a flat sheet 230 that is two-dimensional, a molded sheets/constructs 240 that provide a hand in glove fit for tissue reinforcement, breast implants or other medical devices, anatomy or physiology within the breast (e.g., breast pocket), other organs, or other anatomy, or a “ready-to-use” lumpectomy defect implant 250 within the breast (e.g., breast pocket), other organs, or other anatomy. The solid, implant-style construct can be printed, for example, from a range of from about 20 g to about 4500 g sizes for breast applications (e.g., lumpectomy) or custom sized for other anatomic applications. For example, the implant can be a prolate spheroid shaped (i.e. football) (as exemplary illustrated in FIG. 2), custom shaped, or shaped in another pre-determined geometrical configuration. See above for further discussion on the advantages of a 3D construct, and bioscaffold implants in accordance with various embodiments herein, versus current cadaveric acellular dermal matrix (ADMs) offerings.

Returning to FIG. 5, alternatively, computing device 510 can further include a clinical inputs engine 550 configured and arranged to receive medical data for a specific patient, wherein the implant configuration engine 530 is configured and arranged to generate bioscaffold implant design criteria based on the received medical data for the specific patient. Here, rather than simply receiving design criteria from an outside source, such as a user input, memory storage, and so on, implant configuration engine 530 would personalize the design criteria based on the medical data received. As such, an even more robust implant configuration engine 530 would be provided, which uses data such as patient medical data to provide the requisite, personalized design criteria needed for the image translation engine 540 to generate a bioscaffold implant image that meets that generated design criteria.

In various embodiments, the implant configuration engine 530 can also take an image of a void volume based on the surface topography of the resected portion. The implant configuration engine 530 may also determine a surface topography of the void volume on the image of the tissue section. The implant configuration engine 530 may also generate bioscaffold implant design criteria based on a modification factor determined from the received treatment data and medical data for the specific patient.

According to aspects, the modification factor may include a mathematical model. According to aspects, the medical data may include a medical history of the specific patient. For example, the medical history may include an age of the specific patient or an age of the void volume.

According to aspects, the treatment data may include a therapeutic regimen. For example, the therapeutic regimen may include past and future chemotherapy or radiation therapy. It is understood that other therapies and/or treatments that change a morphological nature of tissue may also be included.

System 500 can further include an image capture device 560. In accordance with various embodiments, the image can be two-dimensional. The image can also be three-dimensional. The imaging scans can originate from any source known in the art. Examples include, but are not limited to, magnetic resonance imaging (MRI), computed tomography scan (CT scan), basic x-ray, ultrasound, nuclear medicine imaging (including positron-emission tomography [PET]), nuclear medicine, elastography, tomography, echocardiography, functional near-infrared spectroscopy, magnetic particle imaging, photographs, etc.

The image captured by image capture device 560 can be stored in a physical storage unit 570 such as a local computer, attached memory storage unit, or remote computer or storage unit connected via to device 560 though standard data transfer means (discussed below). Image data can then be transferred from unit 570 to computing device 510 for processing. Unit 570 can also be configured and arranged to receive and store patient medical data to also be transferred to computing device 510 for processing when prompted. As should be apparent, all such devices and units can be equipped with associated input devices for user inputs of data via known methods such as key entry, portable memory (e.g., memory stick), and so on. For example, patient medical data can be entered at an input device associated with image capture device 560, or directly at physical storage unit 570, in such unit possesses an associated input device.

In accordance with various embodiments, image data, patient medical data, or both sets of data can be transferred, either directly from image capture device 560, or via storage unit 570 to a cloud server 580 via standard data transfer means. Image data and/or patient data can then be transferred from cloud server 580 to computing device 510 for processing.

Regarding medical data for a specific patient, in accordance with various embodiments, the medical data can include, for example, patient demographics, procedure type, surgical information, medical history, patient physical data, and combinations thereof.

Patient demographic data can include many various types of patient specific demographic data. This data can include, for example, patient age, height, and bodyweight, BMI, race, etc.

Procedure type data can include many various types of patient specific procedure related data, with the data collected capable of being adjusted depending on the base procedure desired. For example, types of procedures can include primary breast augmentation, revision augmentation, primary reconstruction, revision reconstruction or lumpectomy. Surgery type data can include, for example, breast reduction, pocket adjustment, skin adjustment, scar revision, etc. If a reconstruction is desired, further data can include, for example, choice between delayed or immediate procedure, choice between direct to implant (single stage) or a two-stage procedure, consideration of any other therapies in and around the time of the procedure (radiation, chemotherapy, hormone, or other relevant procedure), and considerations if a mastectomy is desired (weight, type and flap use). Location of procedure (left or right breast, for example) can be relevant. Necessity of additional procedures (e.g., mastopexy, etc.) can be also relevant. The procedure type data can also include identifying the necessity for specific devices for certain particular medical events such as, for example, asymmetry, breast tissue atrophy, chest wall deformity, etc.

Surgical information data can include many types of data including, for example, incision size, incision location (e.g., inframammary, transaxially, periareolar, mastectomy scar, etc.), and device placement (e.g., submuscular, subglandular, etc.).

Medical history data can include many types of data including, for example, noted conditions that can compromise or complicate wound healing, smoking status, diabetes status, immune status, hypertension, high cholesterol, OBGYN history (breast cancer, familial breast cancer, related pathological details, etc.), noted demonstrated patient tissue characteristics that are incompatible, noted potential unwanted surgical risk related to treatment for a condition, previous device history (e.g., noted previous implant, implant type—e.g., acellular dermal matrix or synthetic, previous expander), etc.

Patient physical data can include many types of data including, for example, which breast to be operated on, any noted anatomic or physiological abnormalities, current breast size, expected breast size, additional pre-op measurements (e.g., not part of the CT scan), etc.

Regarding design criteria, in accordance with various embodiments, the design criteria can include, for example, biological parameters, structural parameters, mechanical parameters, and combinations thereof.

In accordance with various embodiments, the design criteria can comprise biological parameters. The biological parameters can include, for example but not limited to, cellular infiltration or attachment type, collagen synthesis type, vascularization type, incorporation profile, and combinations thereof. In accordance with various embodiments, the biological parameters can comprise attachment type. The biological parameters can comprise collagen synthesis type. The biological parameters can comprise vascularization type. The biological parameters can comprise incorporation profile.

In accordance with various embodiments, the design criteria can comprise structural parameters. The structural parameters can include, for example but not limited to, protein composition, protein concentration, surface topography, pore size, filament size, construct thickness, fenestration count, hydration level, macro configuration and combinations thereof.

In accordance with various embodiments, the structural parameters can comprise protein composition. The protein composition can comprise Collagen I and Collagen III. The protein concentration can include a Collagen I to Collagen III ratio similar to those contained within human dermis (e.g., fetal, adolescent, adult and elderly) as shown in Table I (see above).

That is, in various embodiments, the protein composition can have a Collagen I to Collagen III ratio in the range of between about 0.5 to about 3.5, or in the range of between about 0.75 to about 3.0, or in the preferred range of between about 0.9 to about 2.5. The preferred Collagen I to Collagen III ratio range of between about 0.9 to about 2.5 may offer advantages against conventional tissue offerings, which are predominantly taken from adult and elderly donors, contain a high ratio (i.e., greater than about 2.4) of Collagen I to Collagen III, thereby providing decreased support, cushioning, protection, reinforcement and covering than do tissues containing lower ratios.

More specific ratios of Collagen I to Collagen III can include about 0.5, about 0.55, about 0.6, about 0.65, about 0.7, about 0.75, about 0.8, about 0.85, about 0.9, about 0.95, about 1.0, about 1.05, about 1.1, about 1.15, about 1.2, about 1.25, about 1.3, about 1.35, about 1.4, about 1.45, about 1.5, about 1.55, about 1.6, about 1.65, about 1.7, about 1.75, about 1.8, about 1.85, about 1.9, about 1.95, about 2.0, about 2.05, about 2.1, about 2.15, about 2.2, about 2.25, about 2.3, about 2.35, about 2.4, about 2.45, about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, about 3.3, about 2.35, about 3.4, about 3.45, about 3.5 and ranges between any two of these values.

In accordance with various embodiments, the structural parameters can comprise surface topography. The structural parameters can comprise pore size. The pore size can be between about 100 microns to about 500 microns. In various embodiments, the pore size can be about 50 microns to about 1000 microns, or about 50 microns to about 500 microns, or about 100 microns to about 1000 microns. The structural parameters can comprise filament size. The filament diameter can be less than about 100 microns.

Further, in accordance with various embodiments, the structural parameters can comprise construct thickness. The structural parameters can comprise fenestration count (e.g., fenestration number, orientation and location/arrangement). The structural parameters can comprise macro size/configuration (e.g., size of implant).

In accordance with various embodiments, the design criteria can comprise mechanical parameters. The mechanical parameters can include, for example but not limited to, tensile strength, stiffness level, max load level, tensile stress, tensile strain, modulus of elasticity, and combinations thereof.

In accordance with various embodiments, the mechanical parameters can comprise tensile strength. The tensile strength can between about 10 to about 200 Newtons per centimeter (N/cm). Further, the tensile strength is between about 30 to about 100 Newtons per centimeter. Preferably, the tensile strength is between about 50 to about 85 Newtons per centimeter.

In accordance with various embodiments, the mechanical parameters can comprise stiffness level. The stiffness level can be less than about 80 Newtons per millimeter. Further, the stiffness level can be less than about 18 Newtons per millimeter. Preferably, the stiffness level can be greater than about 5 Newtons per millimeter and less than about 18 Newtons per millimeter.

In accordance with various embodiments, the mechanical parameters can comprise max load level. The max load level can be greater than about 50 Newtons. Further, the max load level can be greater than about 150 Newtons. Preferably, the max load level can be greater than about 150 Newtons and less than 400 Newtons.

In accordance with various embodiments, the mechanical parameters comprise tensile stress. The tensile stress can be between about 3 to about 100 megapascals. Preferably, the tensile stress can be between about 10 to about 30 megapascals.

In accordance with various embodiments, the mechanical parameters comprise tensile strain. The tensile strain can be greater than about 10 percent. Further, the tensile strain can be greater than about 35 percent. Preferably, the tensile strain can be from about 20 to about 80 percent.

In accordance with various embodiments, the mechanical parameters comprise modulus of elasticity. The modulus of elasticity can be from about 10 to about 450 megapascals. Further, the modulus of elasticity can be less than about 150 megapascals. Preferably, the modulus of elasticity can be from about 60 to about 150 megapascals.

As discussed previously, by being bioprinted using controlled parameters of the scaffold to meet required properties of the end-product bioscaffold implant (e.g., design criteria), the bioprinted scaffolds allow for personalized medical applications that ADMs simply do not. These personalized medical applications include, for example but not limited to, lumpectomies (as discussed above), tubular cartilage applications, valvular heart disease applications, and coronary artery disease applications, hernia repair applications, tissue graft applications, venous, arterial and lymphatic vessel applications, structural applications or supportive applications where soft tissue defects exist. See above for further discussion on various uses and advantages in the area of tissue reinforcement for bioprinted scaffolds, in accordance with various embodiments, versus currently available tissue offerings.

By controlling the combination of components in the scaffold to provide specific physical properties and implant outcomes, in accordance with the various embodiments herein, the construct will have consistent surface topography throughout with an engineered microarchitecture (controlling microarchitecture properties such as, for example, porosity, fiber diameter, spacing, height of matrix, fiber orientation, etc.). This will provide the appropriate scaffold for a robust wound healing, regenerative, infiltrative and remodeling response.

Additive manufacturing processes, such as, for example, 3D printing manufacturing methods, allow for control of the macro (overall finished shape) and micro (cell units) structure of the construct, as discussed previously. These processes, combined with sophisticated software methods that can incorporate specific design criteria and specific medical data of a patient, provide systems that allow for even greater flexibility and accurate production of bioscaffold implants personalized for specific patients.

Returning to system 500 of FIG. 3, as illustrated, computing device 510 of system 500 can be communicatively connected to image capture device 560 and 3D printer 520. It should be appreciated that each component depicted as part of computing device 510 (and described herein) can be implemented as hardware, firmware, software, or any combination thereof.

In accordance with various embodiments, the computing device 510 can be implemented as an integrated instrument system assembly with image capture device 560 or with 3D printer 520. That is, computing device 510 and image capture device 560 can be housed in the same housing assembly and communicate via conventional device/component connection means (e.g. serial bus, optical cabling, electrical cabling, etc.). Similarly, computing device 510 and 3D printer 520 can be housed in the same housing assembly and communicate via conventional device/component connection means (e.g. serial bus, optical cabling, electrical cabling, etc.).

In accordance with various embodiments, computing device 510 can be implemented as a standalone-computing device (as illustrated in FIG. 5). Computing device 510 can be communicatively connected to image capture device 560 via an optical, serial port, network or modem connection. For example, image capture device 560 can be connected via a LAN or WAN connection that allows for the transmission of imaging data acquired by image capture device 560 to the computing device 510 for analysis. Similarly, and as illustrated, computing device 510 can be communicatively connected to image capture device 560 via a LAN or WAN connection through physical storage 570, cloud server 580, or both.

Further, computing device 510 can be communicatively connected to 3D printer 520 via an optical, serial port, network or modem connection. For example, 3D printer 520 can be connected via a LAN or WAN connection that allows for the transmission of imaging data generated by computing device 510 to 3D printer 520 for production.

In accordance with various embodiments, the functions of computing device 510 can be implemented on a distributed network of shared computer processing resources (such as a cloud computing network) that is communicatively connected to the image capture device 560 and/or 3D printer 520 via a WAN (or equivalent) connection. For example, the functionalities of computing device 510 can be divided up to be implemented in one or more computing nodes on a cloud processing service such as AMAZON WEB SERVICES™.

As described herein, all or certain functions described herein for system 500 components such as, for example, image capture device 560 and computing device 510, can be implemented using computer system 100 as a standalone device or on a distributed network of shared computer processing resources such as a cloud-computing network. Moreover, all or certain functions of specific components of computing device 510 such as, for example, clinical inputs engine 550, implant configuration engine 530, and/or image translation engine 540, can be implemented using computer system 100 as a standalone device or on a distributed network of shared computer processing resources such as a cloud-computing network. It should be understood that the functions of the specific components of the computing device 510 can be implemented via hardware or software. Moreover, the functionalities of each component may be further divided into additional components or collapsed together into less components.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. This disclosure also relates to a device or an apparatus for performing these operations. The systems and methods described herein can be specially constructed for the required purposes, such as the cloud computing network discussed above, or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

The embodiments described herein can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical and non-optical data storage devices. The computer readable medium can also be distributed over a network coupled computer systems so that the computer readable code is stored and executed in a distributed fashion.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical and non-optical data storage devices. The computer readable medium can also be distributed over a network coupled computer systems so that the computer readable code is stored and executed in a distributed fashion.

Although specific embodiments and applications of the invention have been described in this specification, these embodiments and applications are exemplary only, and many variations are possible.

1. A method of manufacturing a bioscaffold implant for a specific patient, comprising:
obtaining an image of a tissue section of the specific patient from imaging scans of the tissue section, wherein the tissue section includes a resected portion;
determining on the image of the tissue section a surface topography of the resected portion;
determining an image of a bioscaffold implant that matches the surface topography of the resected portion; and
manufacturing a bioscaffold implant with a surface portion that mirrors the surface topography of the resected portion.

2. The method of manufacturing a bioscaffold implant for a specific patient of claim 1, wherein the image is two-dimensional.

3. The method of manufacturing a bioscaffold implant for a specific patient of claim 1, wherein the image is three-dimensional.

4. The method of manufacturing a bioscaffold implant for a specific patient of claim 1, further including:
receiving design criteria for the bioscaffold implant; and
determining an image of a bioscaffold implant that satisfies the design criteria.

5. The method of manufacturing a bioscaffold implant for a specific patient of claim 1, further including:
receiving medical data for the specific patient,
generating design criteria for the bioscaffold implant based on the received medical data for the specific patient; and
determining an image of a bioscaffold implant that satisfies the design criteria.

6. The method of manufacturing a bioscaffold implant for a specific patient of claim 4, wherein the design criteria is selected from the group consisting of biological parameters, structural parameters, mechanical parameters, and combinations thereof.

7. The method of manufacturing a bioscaffold implant for a specific patient of claim 5, wherein the design criteria is selected from the group consisting of biological parameters, structural parameters, mechanical parameters, and combinations thereof.

8. The method of manufacturing a bioscaffold implant for a specific patient of claim 5, wherein the medical data is selected from the group consisting of patient demographics, procedure type, surgical information, medical history, patient physical data, and combinations thereof.

9. The method of manufacturing a bioscaffold implant for a specific patient of claim 4, wherein the design criteria comprises biological parameters.

10. The method of manufacturing a bioscaffold implant for a specific patient of claim 9, wherein the biological parameters are selected from the group consisting of cellular infiltration or attachment type, collagen synthesis type, vascularization type, incorporation profile, and combinations thereof.

11. The method of manufacturing a bioscaffold implant for a specific patient of claim 9, wherein the biological parameters comprise attachment type.

12. The method of manufacturing a bioscaffold implant for a specific patient of claim 9, wherein the biological parameters comprise collagen synthesis type.

13. The method of manufacturing a bioscaffold implant for a specific patient of claim 9, wherein the biological parameters comprise vascularization type.

14. The method of manufacturing a bioscaffold implant for a specific patient of claim 9, wherein the biological parameters comprise incorporation profile.

15. The method of manufacturing a bioscaffold implant for a specific patient of claim 4, wherein the design criteria comprises structural parameters.

16. The method of manufacturing a bioscaffold implant for a specific patient of claim 15, wherein the structural parameters are selected from the group consisting of protein composition, protein concentration, surface topography, pore size, filament size, construct thickness, fenestration count, hydration level, macro configuration and combinations thereof.

17. The method of manufacturing a bioscaffold implant for a specific patient of claim 15, wherein the structural parameters comprise protein composition.

18. The method of manufacturing a bioscaffold implant for a specific patient of claim 17, wherein the protein composition comprises Collagen I and Collagen III.

19. The method of manufacturing a bioscaffold implant for a specific patient of claim 18, wherein the protein concentration comprises a Collagen I to Collagen III ratio of between about 0.75:1 to about 3.5:1.

20. The method of manufacturing a bioscaffold implant for a specific patient of claim 15, wherein the structural parameters comprise surface topography.

21. The method of manufacturing a bioscaffold implant for a specific patient of claim 15, wherein the structural parameters comprise pore size.

22. The method of manufacturing a bioscaffold implant for a specific patient of claim 21, wherein the pore size is between about 100 microns to about 500 microns.

23. The method of manufacturing a bioscaffold implant for a specific patient of claim 15, wherein the structural parameters comprise filament diameter.

24. The method of manufacturing a bioscaffold implant for a specific patient of claim 23, wherein the filament size is less than about 100 microns.

25. The method of manufacturing a bioscaffold implant for a specific patient of claim 15, wherein the structural parameters comprise construct thickness.

26. The method of manufacturing a bioscaffold implant for a specific patient of claim 15, wherein the structural parameters comprise fenestration count.

27. The method of manufacturing a bioscaffold implant for a specific patient of claim 15, wherein the structural parameters comprise hydration level.

28. The method of manufacturing a bioscaffold implant for a specific patient of claim 15, wherein the structural parameters comprise macro configuration.

29. The method of manufacturing a bioscaffold implant for a specific patient of claim 4, wherein the design criteria comprises mechanical parameters.

30. The method of manufacturing a bioscaffold implant for a specific patient of claim 29, wherein the mechanical parameters are selected from the group consisting of tensile strength, stiffness level, max load level, tensile stress, tensile strain, modulus of elasticity, and combinations thereof.

31. The method of manufacturing a bioscaffold implant for a specific patient of claim 29, wherein the mechanical parameters comprise tensile strength.

32. The method of manufacturing a bioscaffold implant for a specific patient of claim 31, wherein the tensile strength is between about 10 to about 200 Newtons per centimeter.

33. The method of manufacturing a bioscaffold implant for a specific patient of claim 29, wherein the mechanical parameters comprise stiffness level.

34. The method of manufacturing a bioscaffold implant for a specific patient of claim 33, wherein the stiffness level is less than about 80 Newtons per millimeter.

35. The method of manufacturing a bioscaffold implant for a specific patient of claim 29, wherein the mechanical parameters comprise max load level.

36. The method of manufacturing a bioscaffold implant for a specific patient of claim 35, wherein the max load level is greater than about 50 Newtons.

37. The method of manufacturing a bioscaffold implant for a specific patient of claim 29, wherein the mechanical parameters comprise tensile stress.

38. The method of manufacturing a bioscaffold implant for a specific patient of claim 37, wherein the tensile stress is between about 3 to about 100 megapascals.

39. The method of manufacturing a bioscaffold implant for a specific patient of claim 29, wherein the mechanical parameters comprise tensile strain.

40. The method of manufacturing a bioscaffold implant for a specific patient of claim 39, wherein the tensile strain is greater than about 10 percent.

41. The method of manufacturing a bioscaffold implant for a specific patient of claim 29, wherein the mechanical parameters comprise modulus of elasticity.

42. The method of manufacturing a bioscaffold implant for a specific patient of claim 41, wherein the modulus of elasticity is from about 10 to about 450 megapascals.

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. A method of manufacturing a bioscaffold implant for a specific patient, comprising:
obtaining an image of a tissue section of the specific patient from imaging scans of the tissue section, wherein the tissue section includes a resected portion;
determining on the image of the tissue section a surface topography of the resected portion;
determining an image of a void volume of the resected portion that matches the surface topography of the resected portion; and
determining a geometry for a bioscaffold implant based on a modification factor determined from input physiological values or a therapeutic regimen;
manufacturing the bioscaffold implant with a surface portion based on the determined geometry.

125. The method of manufacturing a bioscaffold implant for a specific patient of claim 124, wherein the modification factor comprises a mathematical model.

126. The method of manufacturing a bioscaffold implant for a specific patient of claim 124, wherein the input physiological values comprise a medical history of the specific patient.

127. The method of manufacturing a bioscaffold implant for a specific patient of claim 126, wherein the medical history comprises an age of the specific patient or an age of the void volume.

128. The method of manufacturing a bioscaffold implant for a specific patient of claim 124, wherein the therapeutic regimen comprises past and future chemotherapy or radiation therapy.

129. The method of manufacturing a bioscaffold implant for a specific patient of claim 124, further comprising running a simulation based on historical clinical data to determine the geometry.

130. The method of manufacturing a bioscaffold implant for a specific patient of claim 124, further comprising implanting the bioscaffold implant in the specific patient in an outpatient procedure.

131. (canceled)

132. (canceled)

133. (canceled)

134. (canceled)

135. (canceled)

136. (canceled)
